(19) United States
(12) Patent Application Publication
(10) Pub. No.: US 2021/0220507 A1
(43) Pub. Date: Jul. 22, 2021

(54) DISINFECTION AND DEODORIZATION EQUIPMENT USING UV-A

(71) Applicants: LIGHTING SYSTEMS AND SOLUTIONS PRIVATE LIMITED, Kerala (IN); PALCO CONSULTANTS, Bayreuth (DE)

(72) Inventors: Syriac Joseph PALACKAL, Vaikom (IN); Helmut ALT, Bayreuth (DE); Radhakrishnan Gopalakurup MANIKKUNATH, Ernakulam (IN); Dileep Kumar Krishnan VELAMMAVUKUDY, Ernakulam (IN)

(21) Appl. No.: 16/755,563

(22) PCT Filed: Apr. 12, 2018
(86) PCT No.: PCT/IN2018/000022

§ 371 Date: Apr. 11, 2020

(30) Foreign Application Priority Data
Oct. 13, 2017 (IN) 201741036402

(51) Int. Cl. A61L 9/20; B01J 21/06; B01J 35/00; B01J 35/02

(52) U.S. Cl. CPC A61L 9/205; B01J 21/063; A61L 2209/12; B01J 35/023; A61L 2209/134; B01J 35/004

(57) ABSTRACT

Embodiments of the invention comprise disinfection and deodorization equipment employing ultraviolet light (UV-A) as an energy source in the Photo-catalytic process for cleaning environments where Microbial pathogens or Volatile organic compounds are present causing Hospital Acquired Infections (HAI) or malodors. In one embodiment of the invention, disinfection and deodorizing equipment comprises a Reaction Chamber having an inlet for receiving air and an outlet for discharging clean air by Photo-catalysis process which in turn accelerates the oxidization process in the atmosphere and decomposes any airborne toxic or volatile organic matter. The reaction chamber may further comprise a plurality of segmented baffles with various sizes, shapes and designs.

### FIELD OF THE INVENTION

This invention relates to a unique disinfection and deodorizing equipment that uses the UV-A light as energy source and more particularly, baffle plates coated with photo-catalyst to aid photo-catalysis that accelerates the rate of disinfection and deodorization to clean contaminated air that may contain microbial pathogens and/or Volatile organic compounds.

### SUMMARY OF THE INVENTION

Ultraviolet light (UV-C) light is an effective means for pollutant removal from contaminated atmosphere through either direct UVC photolysis or UVC radiation-indirectly-induced oxidation of chemical compounds. Although UV-C has germicidal properties, it is also harmful to the human it known to cause cancer. Therefore, during operations, it should be ensured that there is no exposure to human. To solve this problem, we have developed a new system using photo-catalyst that can be activated using UV-A light source.

The UV-A serves two purposes like partial disinfection of microbes and activation of photo-catalyst. The light ray's distribution within the disinfection instrument plays a vital role to obtain the desired effectiveness or in activation of the target compounds or microbial pathogens. The UV-A fluence rate is attenuated by the distance from the UV-A lamp and the proximity with the photo-catalyst surface. Generally, the higher the rate of photo-catalysis, faster the deactivation of microbes or Volatile organic compounds.

Developing a suitable flow pattern is an important consideration for increasing the efficiency of a UV-A based disinfection equipment. It is desirable that the flow pattern result in sufficient radial mixing with a uniform residence time so that the photo-catalyst surface receives a relatively uniform UV-A dosage to activate the photocatalyst. Turbulent flow is typically used to achieve sufficient radial mixing.

An ultraviolet light source (UV-A) used in photo-catalyst process for cleaning environments where microbial pathogens are present causing HAI (hospital acquired infections) are disclosed. In one embodiment, a disinfection equipment is disclosed which includes a vessel having an inlet for receiving air and an outlet for discharging. Photo-catalyst titanium dioxide which in turn accelerates the oxidisation process in the atmosphere and decomposes any airborne toxic organic matter.

The disinfection/deodorization chamber further includes a plurality of segmented baffles with various sizes, shapes and designs

### BACKGROUND OF THE INVENTION

At present harsh chemicals (like chlorine containing compounds, ozone, hydrogen peroxide, peroxy acid, formaldehyde . . . ) are used for a thorough cleaning of the hospital. In severe cases, even the room must be evacuated, sealed and fumigated. Although the microbes are eliminated by fumigation, as soon as an infected patient enters the room, the microbes spreads in the room. As the microbe concentration fluctuates widely between cleaning intervals, if possible it is preferred to continuously clean the room.

### PRIOR ART

Unfortunately, although many claims to have developed equipment to address this challenge, as far none of them stood to their promises and the hospitals continue to use fumigation. Other than fumigation, three technologies that had been proposed since decades for this type of application are: 1. HEPA filter, 2. UV-C, 3. Photo-catalyst or a combination thereof.

Although HEPA filter is adequate in removing the microbes and dust, extreme care should be given for maintenance of the unit. If the filters are not replaced at proper interval, the microbes collected on the filter will starts multiplying on the filter and acts as a contamination/infection source. Determining the proper filter replacement interval is challenging because there is no way to determine the concentration of microbes on the filter.

As UVC is harmful to the human, it is preferable not to use it. Therefore UV-A was looked at to replace the UV-C. The germicidal property of UV-A is very slow acting due to the low energy quantum associated with it compared to the UV-C. It typically takes more than one-hour radiation to get some germicidal property. Therefore, there are no commercial equipment currently operating using UV-A for energy source in germicidal application. We developed a photo-catalyst reaction chamber that can utilize the low energy quantum from the UV-A to eliminate microbes.

Yet another factor that need to be considered is the ozone production during UV-C radiation. It is a settled position that when oxygen in the air can be converted to ozone when it irradiated with UVC light. This ozone is a poison and although the air gets disinfected, it will also contain ozone—the microorganism gets killed, but a poison is added to the air. As UV-A cannot produce ozone, this danger is also eliminated. Therefore, it is preferred to use UV-A light as energy source instead of UV-C.

### DESCRIPTION OF THE INVENTION

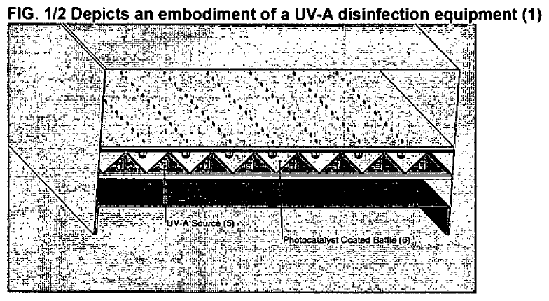

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass direct and indirect mountings, connections, supports, and couplings. Further, “connected” and “coupled” are not restricted to physical or mechanical connections or couplings. In the description below, like reference numerals and labels are used to describe the same, similar or corresponding parts in the several views of FIGS. 1-.2

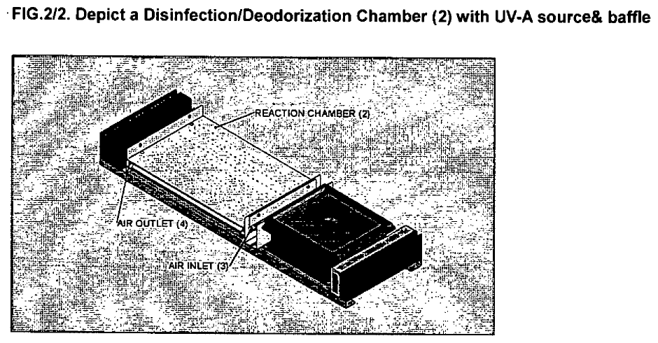

Referring to FIG. 2, an embodiment of a Disinfection equipment 1 in accordance with the present invention is shown as a partial cross-sectional view. The disinfection equipment 1 includes a reaction chamber (2) (design of the reaction chamber can vary)—having an air intake opening (3)—and a discharge opening which is the air outlet (4)—The reaction chamber is fabricated with a UV-A reflective material preferably, Aluminium and may be used for an advanced oxidation process. The size and shape of the reaction chamber is related to the volume and size of contaminated room where the microbial pathogens are present, and based on the UV-A output

To increase input UV-A energy, more number of UV-A lamps or lamps with higher output are placed along the reaction chamber, although it is understood that other configurations may be used. The UV-A source may be tube or LED.

The UV-A based disinfection equipment (1) further includes baffles which extend around the UV-A lamps erected along the reaction chamber of the Disinfection equipment. In a preferred embodiment, the baffles serve to guide or channel UV-A light and air a flow path which corresponds to the shape of the respective baffles designs as air passes through the intake opening to the discharge end of the reaction chamber. The baffles are made of the UV-A reflective material preferably Aluminium and coated with a photo-catalyst preferably titanium dioxide

### Equipment Testing

a. Single pass test

The disinfection/deodorization equipment with various configurations were evaluated for efficacy of the air disinfection using the single pass test where the air pass only once through the equipment. The microbial load (bacteria/fungi) which is measured as Colony Forming Units (CFU) in the ambient air estimated by allowing the air to impinge onto a nutrient agar plate held at the inlet of machine (named as control plate). The microbial load in the air that has passed through the machine is estimated by allowing the air from outlet of the machine to impinge onto another nutrient agar plate held at the outlet. The duration of tests were 15 minutes. It was found that the bacterial load is inactivated completely after going through the machine. The counts were taken several times to confirm the data in the Table 1 below.

TABLE 1
Testing Disinfection Capability of Equipment

| | | Inlet | Outlet | Microbe |
|---|---|---|---|---|
| | Equipment | CFU count | CFU count | reduction (%) |
| 1 | VB-15 RG-90-S | 3 | 0 | 100% |
| 2 | VB-15 RG-90-D | 3 | 0 | 100% |
| 3 | VB-30 RG-125-S | 7 | 0 | 100% |
| 4 | VB-30 RG-160-S | TNTC* | 0 | 100% |

*Too NumerousTo Count

The above tests demonstrate that the microbial colonies are totally eliminated when the air passes just once through the equipment. The best known scientific literature on disinfection property of UV-A from M. Gademoulastates that it takes about 70 minutes to achieve a log 3 reduction in the microbial count. Our equipment achieves even better disinfection (log 6) in 0.156 seconds, which is 25000 times faster than the best-known scientific literature.

b. Room Disinfection

In this series of tests, the equipment was operated in a closed room to determine how much the microbial load in the room reduces after 6 hours of operation. APHA 2001, Edition 4 Chapter 3 protocol was used for the tests. CFU in the room is measured using agar plate at certain intervals. The data is given in the table 2 below:

TABLE 2
Room Disinfection Testing

| | | | | Microbe |
|---|---|---|---|---|
| | Equipment | Before CFU | After 6h CFU | reduction (%) |
| 1 | VB-15 RG-90-S | 20 | 0 | 100% |

The above data shows that the microbial count in the room was nil—a total elimination of microbes achieved Below 15 CFU count is considered as clean room.

Odour control using this equipment was tested at various hotels to determine the efficiency to remove organic odours. It was found to be very efficient in removing bad odours. Unfortunately, as we were not able to get any analytical tool to quantify the efficiency of odour removal only a subjective statement can be made.

1. A disinfection and deodorizing device, comprising: baffle plates coated with photo-catalyst to aid photo-catalysis that uses UV-A light as energy source to accelerate the rate of disinfection and deodorization to clean contaminated air that comprise at least one of microbial pathogens and I or VOCs (Volatile Organic Compounds).

2. The device of claim 1 wherein a pattern of light distribution is distributed evenly that enables the UV-A light coming into contact with the baffle plates coated with photocatalysts arranged within a reaction chamber of the disinfection instrument to provide log 5 or above disinfection efficiency of the target compounds or microbial pathogens.

3. The device of claim 1 wherein the UV-A fluence rate is attenuated by the distance from the UV-A lamp and the proximity with the photocatalyst surface.

4. The device of claim 1 wherein developing a suitable flow pattern increases the efficiency of a UV-A based disinfection equipment.

5. The device of claim 1 further comprising a reaction chamber having an air intake opening and a discharge opening which comprises an air outlet.

6. The device of claim 1 wherein to increase input UV-A energy, multiple UV-A lamps or lamps with higher output are placed along the reaction chamber.

7. The device of claim 1 wherein the UV-A based disinfection equipment further includes baffles which extend around the UV-A lamps erected along the reaction chamber of the Disinfection equipment.

8. The device of claim 1 wherein the disinfection equipment includes a chamber having an inlet for receiving air and an outlet for discharging Photocatalyst titanium dioxide which in turn accelerates the oxidation process in the atmosphere and decomposes any airborne toxic organic matter.

9. The device of claim 1 wherein the photo-catalyst comprises Anatase, nano-TiO2.

10. The device of claim 4 wherein the flow pattern results in sufficient radial mixing with a uniform residence time so that the photo-catalyst surface receives a relatively uniform UV-A dosage.

11. The device of claim 10 wherein sufficient radial mixing comprises turbulent flow.

12. The device of claim 5 wherein the reaction chamber comprises a UV-A reflective material.

13. The device of claim 12 wherein the reflective material comprises Aluminum.

14. The device of claim 6 wherein the UV-A source comprises at least one of a tube or LED.

15. The device of claim 7 wherein the baffles channel UV-A light and air flow along a path corresponding to a shape of the baffles as air passes through the intake opening to the discharge end of the reaction chamber.

16. The device of claim 15 wherein the baffles comprise a UV-A reflective material.

17. The device of claim 16 wherein the reflective material comprises Aluminum coated with a photo-catalyst preferably titanium dioxide.

18. The device of claim 8 wherein the chamber further comprises a plurality of segmented baffles.
